(19) United States
(12) Patent Application Publication
(10) Pub. No.: US 2008/0262428 A1
(43) Pub. Date: Oct. 23, 2008

(54) METHOD FOR VAP PREVENTATIVE VENTILATION OF INTUBATED CRITICALLY ILL PATIENTS

(75) Inventor: Fred G. Gobel, Wilhemsfeld (DE)

Correspondence Address: DORITY & MANNING, P.A., POST OFFICE BOX 1449, GREENVILLE, SC, 29602-1449, US

(73) Assignee: KIMBERLY-CLARK WORLDWIDE, INC., Neenah, WI (US)

(21) Appl. No.: 11/736,816

(22) Filed: Apr. 18, 2007

(51) Int. Cl. A61M 37/00

(52) U.S. Cl. 604/150

(57) ABSTRACT

An intermittent, large-volume lavage/suctioning scheme is provided. A lavage solution is first applied into the subglottic cavity, in sufficient volume to mechanically dislodge and wash-out pooled contaminated solid materials from the oro-pharyngeal spaces, the naso-pharyngeal spaces and the sinuses adjunct to the intra-nasal cavities. The dislodged contaminated solid materials are removed with large bore suctioning tools suited for atraumatic, deep naso-pharyngeal and oro-pharyngeal insertion. After the suctioning procedure has been performed, a space filling gel seal that is composed of a self-degrading physiologic substance can be installed within the subglottic space and subsequently washed out into the pharynx with the next lavage procedure. The lavage/suctioning scheme largely reduces or eliminates a bacteria colonization pattern where VAP causing pathogens have been.

### CROSS-REFERENCE TO RELATED APPLICATIONS

N/A

### STATEMENT REGARDING FEDERALLY SPONSORED RESEARCH OR DEVELOPMENT

N/A

### BACKGROUND

The present disclosure relates to methods for reducing the incidence of ventilator associated pneumonia (VAP) in intubated and mechanically ventilated patients.

A mechanically ventilated patient undergoes so-called positive pressure ventilation (PPV), whereby the patient's lungs are actively inflated by a mechanical ventilator to a certain tidal volume or distal airway pressure. A mechanically ventilated patient typically would have a tube inserted into the trachea (tracheal tube or tracheotomy tube). The proximal end of the tube is connected to a ventilator to introduce air into the lungs and assist the patient's breathing. The distal end of the tube is disposed below the glottis and above where the trachea branches to the bronchial tubes. A cuff mechanically secures the distal end of the tube at this location within the trachea. The space inside the trachea between the cuff and the glottis is called the sub-glottic space.

The cuff is a thin-walled, sleeve-like member that surrounds a section of the distal end of the tube and occupies the space between the exterior of that section of the distal end of the tube and the walls of the trachea. The cuff typically is inflated and pressurized via a lumen formed in the wall of the tube. One end of the lumen is disposed outside the patient and connected to a source that maintains pressure in the cuff. The other end of the lumen communicates with the interior of the cuff to inflate the cuff outwardly against the interior walls of the trachea.

The inflated cuff also functions to create a seal between the tube's exterior and the interior surfaces of the walls of the trachea. The cuff's sealing function enables the performance of so-called positive pressure ventilation. The cuff's sealing function also is intended to prevent liquids or other pharyngeal material from being aspirated into the broncho-pulmonary airway.

With the cuff in place, secretions containing pathogens tend to pool above the cuff in the sub-glottic space. While state of the art tracheal tube cuffs provide sufficient seal performance for positive pressure ventilation, the seal against such secretions is not sufficient to completely prevent the descent of VAP causing bacteria into the distal trachea and into the broncho-pulmonary airway. Axially directed movement of the tracheal tube inside the trachea may cause downward directed dislocation of the tracheal tube cuff within the trachea to such a degree that pathogenic bacteria colonizing such pooled secretions may be introduced into the distal portions of the trachea and cause broncho-pulmonary inflammation. Further, rapid and significant changes of intra-thoracic pressure may occur. The changes may be induced by the patient's chest apparatus during extreme respiratory maneuvers, as in for example deep inspiration after a series of coughs, or by rapid inspiration under insufficiently low flow provided by the ventilator. Additionally, changes in intra-thoracic pressure may occur during spontaneous breathing or during so called supported/assisted mechanical ventilation. Each of these patient induced changes of intra-thoracic pressure may reduce the filling pressure of the tracheal cuff to a degree that colonized pharyngeal secretions can get past the cuffs seal and intrude into the lower airways.

Ventilator-associated pneumonia results from two major infection mechanisms, so-called exogenous infection and endogenous infection. In the so-called exogenous mechanism of airway contamination, pathogens enter the lower airways from outside of the patient, in most cases via the hose system of the connected ventilator circuit or by intermittently inserted catheters into the trachea and bronchi. The exogenous origin of VAP can be controlled largely by the insertion of filters and appropriate hygienic precautions when opening the ventilator circuit for therapeutic or diagnostic procedures.

In the so-called endogenous mechanism, the VAP-causing pathogen is generated by the patient from pathogens that colonize certain organs and body cavities in a VAP-typical qualitative pattern. These pathogens eventually meet and mix within the pharyngeal cavity. The patient's pharynx and the adjunct cranio-facial cavities as well as the upper GI-tract have been identified by clinical investigators as primary reservoirs for VAP-relevant aspirated bacteria.

In the pathogenesis of VAP in intubated and ventilated patients, tracheal aspiration of colonized pharyngeal secretions that travel past the tracheal tube cuff is commonly being considered the leading cause for the development of broncho-pulmonary airway infection. These pathogens enter the lower respiratory tract mostly via folds within the cuff material that form passages through the cuff of state-of-the-art tracheal tubes. These pathogens enter the lower respiratory tract during intermittent axial dislocations of the cuff due to patient or tube movement or by intermittent phases of total cuff pressure loss. Thus, most investigators consider the endogenous mechanism of infection to be the leading cause of VAP development.

The clinical prevention of VAP has focused on reducing the density of endogenous, patient indwelling VAP-relevant pathogens that otherwise would be present in the pharynx and the upper gastro-intestinal organs. Presently, the most effective approach is antibiotic eradication of bacteria, known as SOD, SGD or, SDD. SOD means Selective Oral Decontamination, which calls for topical application of non-resorbable antimicrobial agents to accessible surfaces of the oral cavity. For example, U.S. Patent Application Publication No. 2003/0073625 discloses a method of preventing VAP that topically administers an AntiBiotic-367 peptide to the oral cavity of an intubated patient. The composition is applied directly to accessible surfaces of the oral cavity with likely contact to portions of the endotracheal tube. At times attempts are made to remove the applied the composition by various cleaning techniques that include rinsing away any removable composition from contacted surfaces. The CFUs (colony forming units of flora associated with an infection) in the oral cavity of the patient can be conveniently obtained such as by swab testing to monitor the efficacy of the method. When the composition is provided as a rinse, the aid of a sprayer can be used. The composition also can be applied to the accessible surfaces using an applicator such as a sponge or other soft, absorbent applicator.

SGD means Selective Gastric Decontamination, which means administration of solutions of antimicrobial to the stomach by swallowing or through a feeding tube. SDD means a combination of both SOD and SGD to effect decontamination of the pre-digestive tract, which includes oro-pharyngeal and gastric regions. However, while perhaps the most effective approach, due to the risk of antibiotic-resistant bacteria which is known to be selecting under preventive antibiotic therapy, decontamination has not entered clinical routine.

The most frequently practiced VAP-preventive measure today is semi-recumbent positioning of the patient's chest in order to favor retention of gastric fluids below the pharynx. This positioning of the patient is combined with the use of so called naso-gastric (NG) tubes for continuous decompression of gastric pressure and drainage of stomach contents. This combination reduces the risk of trans-esophageal reflux of infection inducing pathogens into the patient's pharynx and adjunct cranio-facial cavities.

Another, recently introduced prophylactic measure taken to reduce the incidence of VAP includes subglottic suctioning. This evacuation concept calls for supervised continuous or intermittent suctioning of pharyngeal secretions that have descended into the so-called subglottic space. Such supervised continuous or intermittent suctioning is a procedure that is conducted by a healthcare provider or by an appropriate automated sensing-monitoring system. Risks associated with performing this procedure include: aspiration of subglottic tissue into the suctioning opening; occlusion of the suctioning shaft integrated suctioning lumen due to solidifying secretions; and damage of the larynx caused by the prolonged flow of dry air and the associated drying out of the larynx tissue that is exposed to this flow of dry air.

Further VAP-preventive measures that have been established in clinical routine are pH adjustment in gastric secretions, pharmacological support of esophageal sphincter function and stomach motility, as well as frequent oral care. The various strategies to influence pharyngeal colonization quantity and quality can be shown having certain VAP-preventive potential individually practiced or in combined use. Yet, state of the art naso-gastric (NG) tubes, which are standard care in mechanically ventilated patients, are known to decompress the abdomen insufficiently and produce permanent or short term pressure gradients between the abdominal and thoracic compartments. Such pressure gradients can cause considerable trans-esophageal accession of secretions from the stomach into the pharyngeal spaces, in many cases, thereby facilitating trans-esophageal communication of pharyngeal and intestinal secretions, rather than reducing or preventing it. Furthermore, material contaminated with VAP-inducing pathogens is known to be pooling in the remote cranio-facial cavities, the so called para-nasal sinuses. While secretions trapped in the para-nasal sinuses are known to be a major source of VAP relevant bacteria, such secretions cannot be sanitized with any of the presently used preventive measures.

On the whole, current VAP preventive strategies may be considered inadequate to sufficiently reduce or control pharyngeal colonization with endogenous pathogens. The associated risk of aspiration that produces broncho-pulmonary inflammation remains an unsolved problem in modern Intensive Care Unit (ICU) ventilation. Depending on the specific patient population, incidences of VAP on the order of 10% to 80% of the population are still being reported in the medical literature. Thus, conventional prophylactic approaches that are used in attempts to prevent pharyngeal pathogens from entering the pulmonary tract still allow for substantial occurrences of VAP.

### SUMMARY

Based on current tracheal intubation and esophageal decompression technology, broncho-pulmonary descent of pathogens may be considered a fact in all ventilated patients, and therefore alternative, more effective approaches are needed to reduce the occurrence of VAP. The present disclosure provides such a preventive concept.

In accordance with the present disclosure, an intermittent, large-volume lavage/suctioning scheme is provided. First the pharynx is segmented off as a separated compartment to achieve a continuous, non-irritating, perfusion and functionally organ compatible separation of airway and digestive tract. This desirably is accomplished by combined use of a cuff on a special tracheal ventilation tube and a cuff on a special decompressing naso-gastric (NG) tube. Then a high volume lavage is performed by introducing a lavage solution into the subglottic and/or the hypopharyngeal space. This is the portion of the created space/compartment that is located closest to the tracheal tube cuff (subglottic larynx/trachea or hypopharynx). The lavage solution is supplied in sufficient volume to fill not only the oro-pharyngeal and the naso-pharyngeal spaces, but also the oral and nasal cavity, as well as the sinuses adjunct to the nasal cavities. The level of lavage solution within the compartment is gradually increased until all cranio-facial surfaces and cavities (including the frontal sinuses) have been flooded by the lavage solution and the level of solution reaches the nasal and oral openings. The lavage solution is applied into the anatomically deepest portion of the compartment, which is the portion being closest to the cuff that seals the trachea, thereby granting the highest possible surface lavage effect at the very location where aspiration of contaminated material is considered primarily to be taking place.

This high-volume application of the lavage solution effects a dislodgement and mobilization of material residing on all surfaces of the created compartment and a wash-out of pooled, contaminating liquid and solid materials (secretions, stomach content, blood clots, pus) from spaces potentially serving as a reservoir filled with pathogens. These dislodged materials and liquids are washed into the nasal and oral cavities, from where they can be removed with large bore suctioning tools that are suited for atraumatic, deep naso- and oro-pharyngeal insertion. Most of the lavage solution desirably is removed by suctioning catheter/catheters that reach atraumatically into the deepest portions of the naso-pharyngeal and hypopharyngeal portion of the compartment. Removal of possibly remaining solution from the section of the compartment above or adjacent to the airway seal can be done by connecting the lavage solution supply line of the tracheal tube/NG tube to the suctioning line. Being repeated in daily or shift intervals, the suggested lavage/suctioning scheme can keep the mucosal surfaces in a state of significantly reduced bacterial colonization.

A tracheal tube/tracheotomy tube that is single-cuffed or double-cuffed with a particular low-pressure sealing cuff material is used. The tube includes a shaft integrated lumen that supplies a respective subglottic/hypo-pharyngeal lavage/suction port. An NG tube is also used with a particular designed esophageal seal element (a balloon with residual wall material) and an optional shaft integrated lavage/suctioning port/opening into the hypo-pharyngeal portion of the created compartment. The lavage solution can be applied via additional ports, which can be channels that are integrated into the shaft of the tracheal ventilation tube and/or the NG-tube.

The seal element of the tracheal tube and the seal element of the NG tube are designed to produce a sufficient seal under installation of maximum lavage solution volume and possible pressure changes caused by patient response to irritation under the delivery or removal of the lavage solution. Thus, the tracheal and esophageal seal elements are designed to meet the seal requirements given by the force exerted by the column of lavage solution installed above them, as well as by a continuously fluctuating filling pressure in the balloon seal as the patient's chest and body movements modulate the pressure being experienced by the cuffs.

After the lavage and removal of lavage solution have been completed, a gel-like substance can be applied in the subglottic space and/or the hypo-pharyngeal space above the respective tracheal and esophageal seal elements. The gel desirably contains one or more antiseptic or bacteriostatic agents in it. The gel is applied so that the gel enters and occludes the passages that exist in the cuffs at the ends of the tracheal tube and the NG tube.

The lavage solution desirably is provided to the user in a container that can be connected to at least one of the lines that deliver lavage solution through the ports in the tracheal tube and the naso-gastric tube.

The method also can include other features such as an intermittent antiseptic oro- and/or naso-pharyngeal spray with accordingly designed insertion piece and nozzle, and sampling for pharyngeal bacteria for determining bacterial quality and colonization density.

The high-volume lavage solution of the present disclosure is primarily salt-based (physiologic salt concentration or slightly elevated salt concentrations to trigger mucosal secretion). The high-volume lavage solution can optionally contain other active ingredients that reduce the bacteria colonization from which VAP develops. Such ingredients would include but not be limited to antibiotics or antiseptics, and other natural or artificial substances with a documented colonization reducing effect. Also to be optionally included in the lavage solution are agents known to have a mucosal care effect, such as artificial mucous, vitamins, or lipids or other agents supporting the integrity of the mucosal barrier.

The space filling gel seal that can be installed within the subglottic space or within the hypopharyngeal portion of the compartment after completing the suctioning procedure and removing remaining lavage solution from that anatomically deepest portion of the created pharyngeal compartment desirably can include a self-degrading physiologic substance. Such gel substance should be composed in a way that it behaves viscously enough not to spread diffusely from the location of initial gel application. The gel desirably is composed in a way that it can be removed at any point of time after delivery from the location around the delivery port, whereby in order to mobilize and wash out the gel from there, a force equivalent to about 50 to about 100 cm of water column exerted by the lavage solution is sufficient. The remains of the gel are washed out into the pharynx and are removed from there by suctioning of the oral and nasal cavity in the course of the succeeding lavage procedure. Suitable gel substances can be based on bacteriostatic sugars such as xylitol or higher concentrated sugars like saccharose or structurally similar sugars for example. Further, natural complex based sugars like honey can be added to the lavage solution. Another suitable gel substance can include a gel based on hyaluronic acid and may undergo an enzyme-triggered phase change from gel form to liquid form, thereby facilitating removal of the gel substance when desired by spontaneous degradation. This aspect is especially useful in case parts of the gel should accidentally reach the tracheo-bronchial tract.

The present disclosure provides a VAP preventive care method that takes a preventive approach, which is safe, non-invasive and easy to integrate into care routine. Pharyngeal care of the present disclosure can essentially replace and upgrade so called “oral care” that currently is being performed on most mechanically ventilated patients. The pharyngeal care of the present disclosure is designed to require equal or less care time than conventional oral care methods, yet sanitizes all cranio-facial reservoirs, instead of being limited to the oral cavity and certain portions of the oro-pharynx.

Additional objects and advantages of the present disclosure will be set forth in part in the description that follows, and in part will be obvious from the description, or may be learned by practice of the present disclosure. The objects and advantages of the present disclosure may be realized and attained by means of the instrumentalities and combinations particularly pointed out in the appended claims.

The accompanying drawings, which are incorporated in and constitute a part of this specification, illustrate at least one presently preferred embodiment of the present disclosure as well as some alternative embodiments. These drawings, together with the description, serve to explain the principles of the present disclosure but by no means are intended to be exhaustive of all of the possible manifestations of the present disclosure.

### BRIEF DESCRIPTION OF THE DRAWINGS

A full and enabling disclosure of the present disclosure, including the best mode thereof to one skilled in the art, is set forth more particularly in the remainder of the specification, including reference to the accompanying figures, in which:

FIG. 1 is a partial cross-sectional view of the anatomy of the human head neck and upper thorax.

FIG. 2 is a frontal representation of the positioning of the para-nasal sinus cavities in the human head.

FIG. 3 is a partial cross-sectional view of the anatomy of the human head neck and upper thorax, used to illustrate a tracheal tube with a double cuff arrangement and hypopharyngeal flushing/suctioning port.

FIG. 4 is showing a schematic drawing of the relationship and ratio between the force exerted by the rinsing fluid volume installed in the pharyngeal compartment, and the required seal forces to be effected by the tracheal, laryngo-tracheal and esophageal seal element.

FIG. 5 is a partial cross-sectional view of the anatomy of the human head neck and upper thorax used to illustrate a subglottic plug.

FIG. 6 is a partial cross-sectional view of the anatomy of the human head, neck and upper thorax used to illustrate a combination of a tracheal seal and naso-gastric esophageal balloon seal.

FIG. 7 includes a partial cross-sectional view of the anatomy of the human head, neck, stomach and upper thorax, schematically illustrates a container for lavage solution.

FIG. 8 schematically illustrates an oro-naso pharyngeal suction catheter.

Repeat use of reference characters in the present specification and drawings is intended to represent the same or analogous features or elements of the present disclosure.

### DETAILED DESCRIPTION

Reference now will be made in detail to the presently preferred embodiments of the present disclosure, one or more examples of which are illustrated in the accompanying drawings. Each example is provided by way of explanation of the present disclosure, which is not restricted to the specifics of the examples. In fact, it will be apparent to those skilled in the art that various modifications and variations can be made in the present disclosure without departing from the scope or spirit of the present disclosure. For instance, features illustrated or described as part of one embodiment, can be used on another embodiment to yield a still further embodiment. Thus, it is intended that the present disclosure cover such modifications and variations as come within the scope of the appended claims and their equivalents.

The following acronyms or phrases will have the meanings ascribed to them hereafter.

Enteral means relating to a method of nutrient delivery where fluid is given directly into the gastrointestinal tract. GI means gastro-intestinal. ICU means intensive care unit. NG means naso-gastric. The so called subglottic space is defined as the space that is created between the vocal cords and the upper (proximal) end of the cuff of an intubated, conventional, single-cuffed tracheal tube.

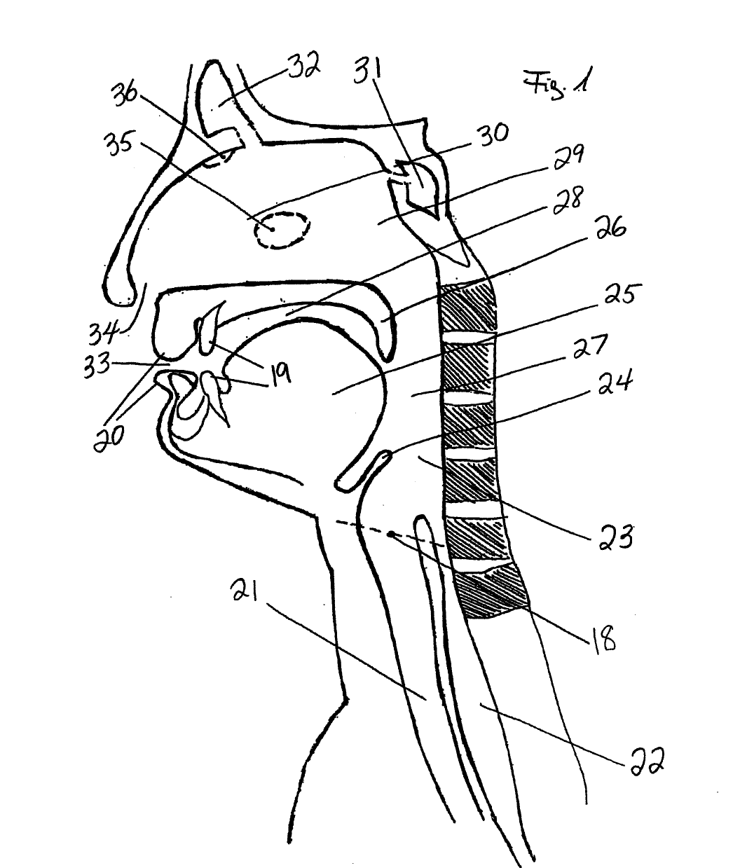

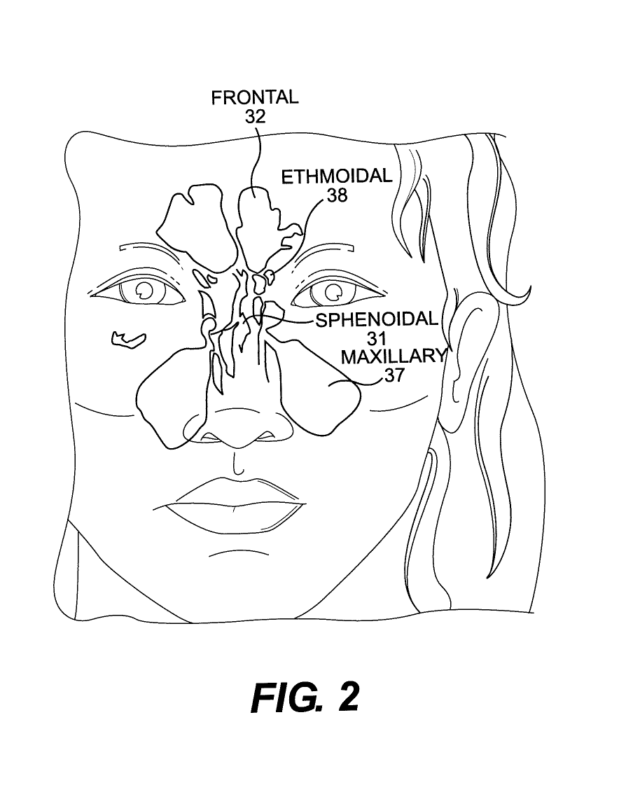

FIG. 1 illustrates teeth 19 and lips 20 and the following portions of the anatomy relevant to the method herein. The trachea 21 forms an airway wherein the level of the vocal cords is generally designated 18, and the esophagus 22 forms the digestive way. The hypo-pharynx 23 is disposed at the crossing point of the airway formed by the trachea 21 and the digestive way formed by the esophagus 22. The epiglottis is designated 24, and the tongue muscle is designated 25. The soft palate is designated 26, and the oro-pharynx is designated 27. The oral cavity is designated 28, and the naso-pharynx is designated 29. The nasal cavity is designated 30, and the spenoidal cavity is designated 31. The frontal cavity is designated 32, and the oral opening is designated 33. The nasal opening is designated 34, the access to maxillary sinus is designated 35, while the access to the ethmoidal sinuses is designated 36. FIG. 2 further illustrates the anatomy of the para-nasal sinuses that include the maxillary sinus 37, ethmoidal sinuses 38, the frontal sinus 32, and the spenoidal sinus 31.

The present disclosure calls for separating the upper respiratory tract (oral and nasal cavity, oro- and nasopharynx, sinuses, as well as hypo-pharynx) from free communication with the upper digestive tract (stomach, esophagus, hypo- and oro-pharynx, as well as oral cavity) via the establishment of a stable space in a manner that results in the isolation of all cranio-facial cavities as a separated compartment.

The created pharyngeal compartment provides a condition to allow for high-volume lavage in the portions of the compartment (subglottic or hypo-pharyngeal space) that are most relevant to the mechanism that produces VAP. A respiratory catheter device (tracheal tube) and a gastro-intestinal catheter device (NG tube) are employed to create the desired compartment. Each of the tracheal tube and NG tube is specifically designed with an inflatable cuff (aka an inflatable balloon seal) to anchor each tube and seal off the organ's lumen. Each of the tracheal tube and NG tube is specifically designed to deploy the lavage solution and to assist in removal of residual lavage solution.

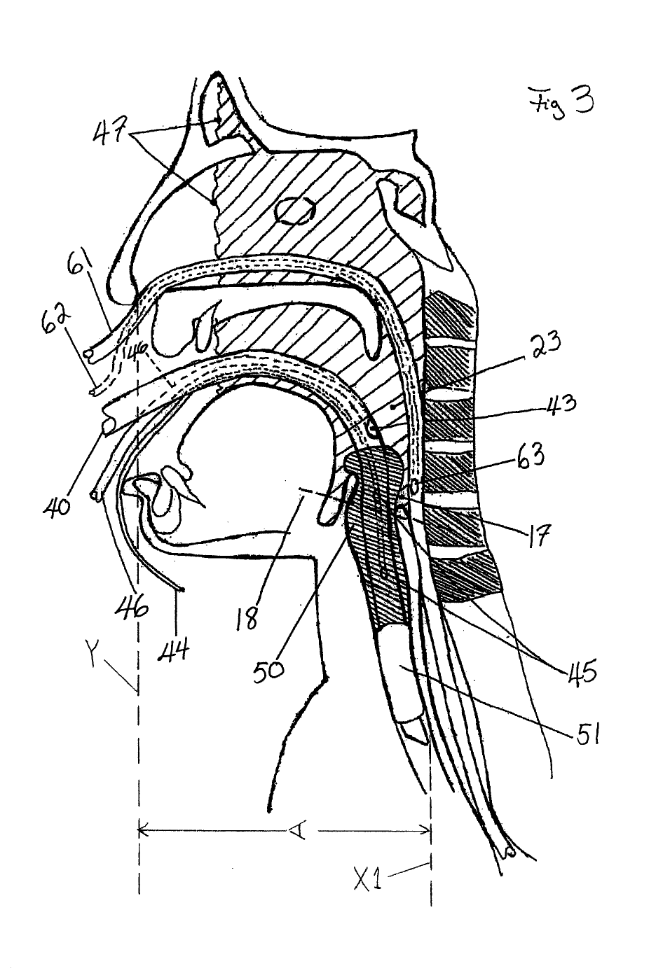

FIG. 3 schematically illustrates a patient that has been intubated with a tracheal tube 40 that is provided with a tracheal tube cuff 51 that defines one boundary of the stable space of the pharyngeal compartment. The tracheal tube 40 also is provided with a wall port 43 that is configured and disposed along tube 40 so as to be positioned to be used for hypopharyngeal lavage and hypopharyngeal suctioning of the lavage solution. The present disclosure desirably uses a tracheal tube 40 whereby the shaft element of the tracheal tube 40 is equipped with an additional lumen 46 that is integrated in the wall of the tube 40 and forms a suctioning and lavage conduit 46 for efficient application of larger volumes of lavage solution. The distal end of the lumen 46 terminates at the wall port 43 that is defined through the wall of the tracheal tube 40.

As shown in FIG. 3, tracheal tube cuff 51 can be surrounded by an additional tamponade element 50. The tube 40 desirably is disposed so that the port 43 opens proximal the tamponating balloon 50 into the hypo-pharyngeal area 23 of the supra-glottic space 17. The tracheal tube 40 desirably is configured so that the tamponating balloon 50 can be inflated via an additional shaft-integrated filling lumen 44 and openings 45 through the wall that defines the tube 40. The tamponade element 50 can be designed to fill out the total subglottic space reaching at least to the level 18 of the vocal cords. Optionally, as shown in FIG. 3 for example, the space filling tamponade 50 can be configured to reach beyond the vocal cord level 18 and into the supraglottic/hypopharyngeal portion of the larynx. However, the port 43 of the lumen 46 that is to supply the lavage solution must be disposed above the top cuff 50 in order to perform the lavage and suction functions required by the present disclosure.

The configuration of a double cuff arrangement secures the best possible seal against the pharyngeally introduced volume of lavage solution. Examples of embodiments of suitable tracheal tubes 40 so configured with double cuff arrangements that provide a space filling tamponade 50 of the subglottic space are described in U.S. Pat. Nos. 6,526,977; 6,745,773 and 6,802,317, which are hereby incorporated herein in their entireties by this reference. As shown in FIG. 3 for example, the subglottic tamponade element 50 can be fully enclosing the distal balloon element 51 completely (cuff 51 inside cuff 50). Alternatively, the two cuffs 50, 51 can be arrayed one after the other on the tube shaft 40 to provide the closest possible approximation of two cuffs so as to prevent formation of secretion retaining gaps in the contact area between the two cuffs.

The step of mechanically separating the respiratory tract from free communication with the digestive tract can include in part using a balloon equipped probe inserted into the stomach for feeding and decompression, wherein the balloon is pressurized to provide an esophageal seal between the walls of the esophagus and the exterior of the probe.

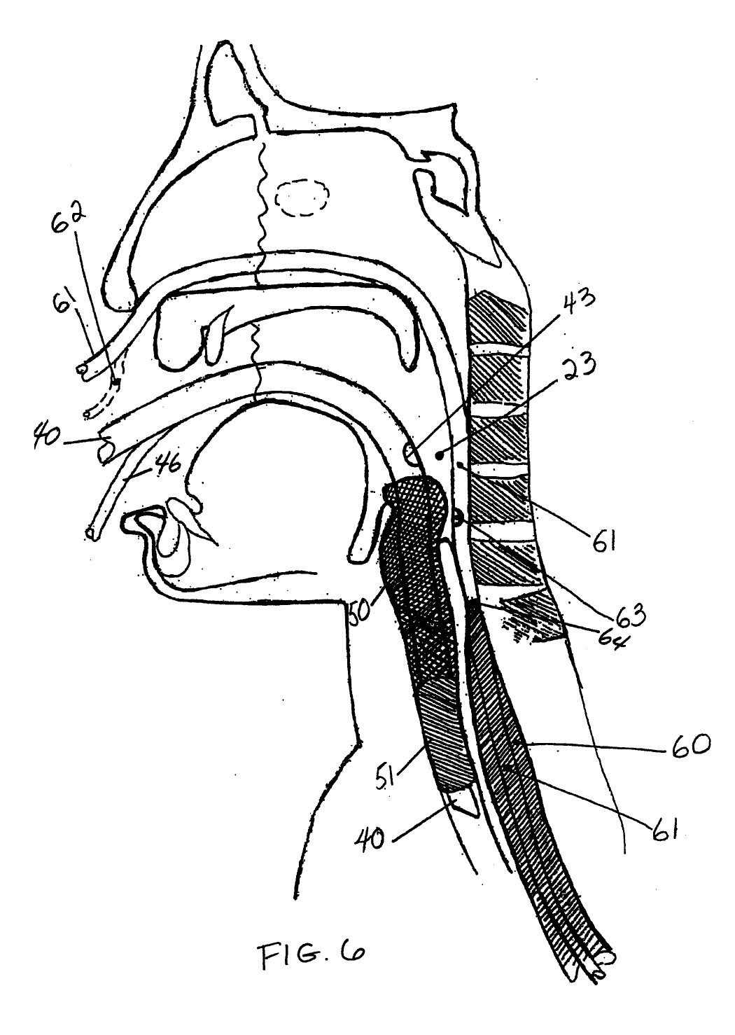

FIG. 6 schematically illustrates the combination of the tracheal seal, which is described above in connection with FIG. 3, and the seal formed by a balloon 60 that is attached to a naso-gastrically applied feeding/decompression tube 61. A naso-gastric tube 61 with a sealing balloon element 60 is described in U.S. Pat. No. 6,551,272, which is hereby incorporated herein in its entirety by this reference. In accordance with an embodiment of the present disclosure, and as shown in FIG. 6 for example, the NG tube 61 can be equipped with an additional lavage/suctioning lumen 62 that opens into the hypo-pharyngeal space 23 via an opening 63. So that a correct anatomical placement of the balloon carrying segment of the NG tube 61 can be determined, the NG tube 61 can be equipped with a radiopaque marker ring 64 at the proximal end of the balloon 60 and a radiopaque marker ring (not shown) at the distal end of the balloon 60.

This naso-gastric tube 61 with sealing balloon element 60 is intended to minimize pharyngeal directed ascension of gastric secretions and/or gastrically applied feeding solution in a ventilated patient. The ascension of gastric contents is known to be facilitated by the rigid shaft structural state of the conventional NG tubes. Such ascension prevents efficient esophageal sphincter closure and permits gastric material to move from the stomach alongside the tube shaft up to the pharynx. The radiopaque marker rings can be used to dispose the sealing balloon element 60 within the esophageal segment between the upper and lower esophageal sphincters. With the sealing balloon element 60 so disposed, gastric material that may ascend in the esophagus can be prevented from moving past the shaft of the NG-tube 61.

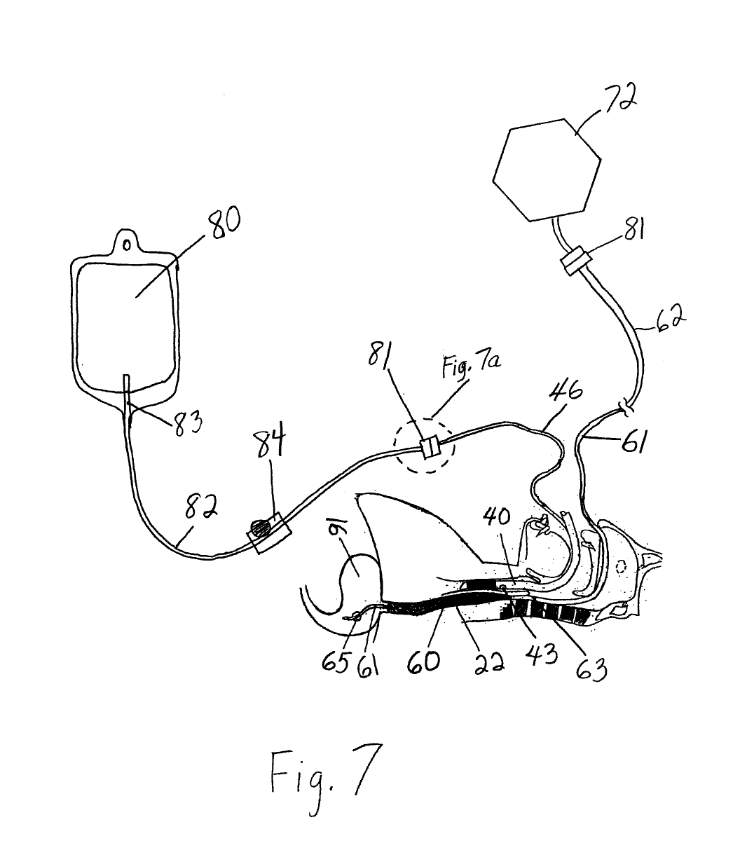

Moreover, so disposed, the damming effect of the sealing balloon element 60 also prevents pharyngeal lavage solution that may descend in the esophagus, from moving past the balloon 60 and descending into the stomach, thus ensuring the attainment of the desired level of lavage solution in the oral and the nasal cavities. By minimizing pharyngeal lavage solution that may descend in the esophagus 22, from moving past the balloon 60 and descending into the stomach 16 (FIG. 7), any resulting increase in gastric volume and pressure that might be caused by the presence of the lavage solution in the stomach is also minimized along with any consequent eventual triggering of gastro-pharyngeal reflux of gastrically applied enteral feeding. The prevention of gastro-pharyngeal reflux during gastrically applied enteral feeding is especially important during the initial treatment days after trauma or surgery, when the patient is generally in a state of deep sedation and the sphincter function of the esophagus may be reduced.

As schematically shown in FIG. 6 for example, in the described double cuffed tracheal tube designs, which integrate a balloon element 51 that fills the subglottic space, the wall port 43 desirably can be positioned directly above the proximal end of the tamponading element 50. Such positioning enables the application of the lavage solution at the anatomically deepest point, namely, the hypo-pharynx 23, which is described in literature as the bacterial pool or bacterial reservoir closest to the tracheal seal and representing the primary source of tracheal aspiration, and therefore is desirably subjected to intensified cleaning.

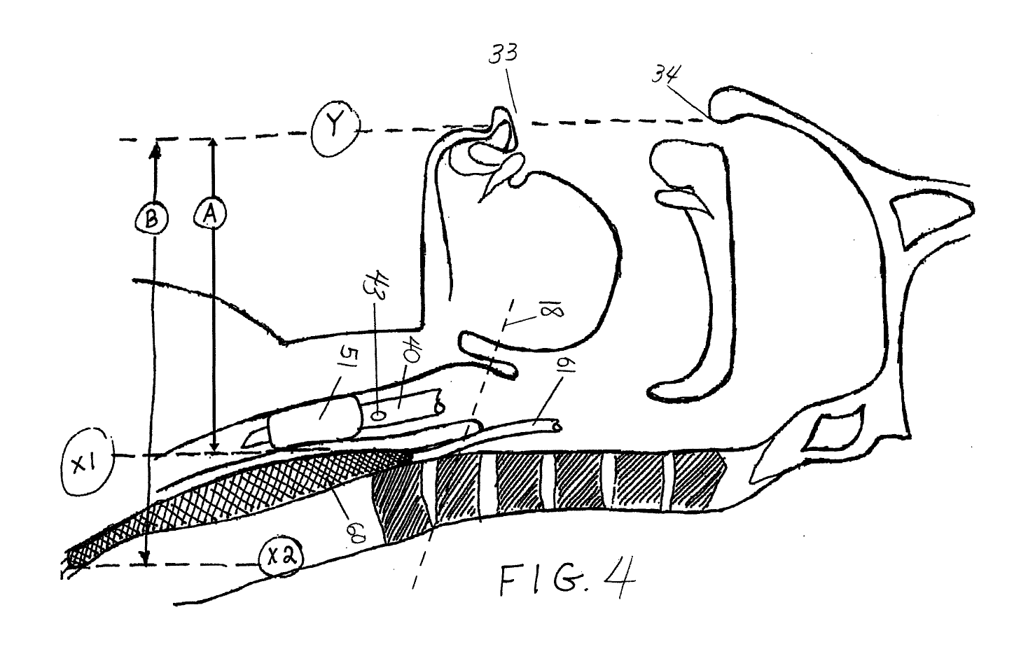

FIG. 4 schematically illustrates the topography of the pharyngeal compartment as it shows itself in the supine positioned patient, who is being intubated with a tracheal tube 40 that has a tracheal sealing balloon element 51 on the distal end of the tube 40. Line X1 defines the compartment's topographically lowest point relevant to the seal provided by balloon element 51. Line Y defines the maximum filling level, which is marked by the connecting line between the mouth opening 33 and the nostrils 34. When flooded with lavage solution from level X1 to level Y, all infection relevant surfaces within the created compartment can be reached by the lavage solution.

In accordance with the principle of application of the lavage solution as close as possible to the tracheal seal 51, which is deemed the primary reservoir for bacterial aspiration, the lavage solution would be installed through a tube shaft integrated port 43 into the space between the proximal end of the cuff 51 and the level 18 of the vocal cords, the so called subglottic space. As shown in FIG. 3 for example, the volume of lavage solution can be infused into the proximal end of the suctioning and lavage conduit 46 and expelled via wall port 43 initially into the hypo-pharynx at a flow rate over a range of about 1.5 to about 3.5 milliliters per second and desirably lavage solution is introduced in a flow that is maintained over a range of about 1.8 to about 2.5 milliliters per second. The lavage solution also can be introduced via port 63 and lumen 62 of the NG tube 61.

The pooled colonized material is washed from the so called subglottic space into the hypo-pharynx and the more rostral compartments. As schematically represented by the widely-spaced, diagonally-hatched area in FIG. 3, the lavage solution can continue to be introduced in this manner. The lavage solution can continue to be supplied until all of the para-nasal spaces have been flooded, which means that the lavage solution should fill a certain anatomical volume that reaches a level that is schematically represented by the dashed line designated Y in FIG. 4. By referring to FIGS. 1 and 2, one may observe that the flushing liquid washes out the hypo-pharynx 23, the oro-pharynx 27, the oral cavity 28, the naso-pharynx 29, the nasal cavity 30, the spenoidal cavity 31, the frontal cavity 32, the maxillary sinus 37 and the ethmoidal sinuses 38. The dashed format of the line Y in FIG. 4 schematically indicates the approximate nature of this specification of the level that desirably is to be attained by the lavage solution. The attainment of this certain anatomical level Y where all of the para-nasal spaces have been flooded begins to be indicated when the lavage solution reaches the level of the nostrils 34 and the corners of the mouth. Such level where all of the para-nasal spaces have been flooded typically will be attained over a range of about one minute to about two minutes.

Lavage Solution

The lavage solution can be based on a physiologic salt concentration. Presently deemed desirable is an elevated salt concentration (beyond 0.9% NaCl) or other salt types or compositions of salts, that trigger mucosal secretion and mucosal self-cleaning.

The lavage solution may be further based on a composition containing xylotol, which is known as having a certain bacteriostatic effect on pharyngeally dwelling bacteria. Alternatively, the lavage solution can be based on a higher concentration of conventional food sugar (e.g. saccharose) or natural honey, which have been described as causing a bacteriostatic effect on wound inflammatory relevant flora. Other bacteriostatic or bactericidal natural agents like tea tree oil (to cite one example) may be contained or provided as alternative solution preparations.

The lavage solution can include antibiotics such as cefotaxime, ceftazidime, cefazolin, cephradine, cefuroxime, ciprofloxacin, vancomycin, tobramycin, ampicillin, piperacillin, carbenicillin, tricarcillin, metronidazole, erythromycin, gentamycin, trimethoprim, clindamycin, tetracycline, tazobactam, linezolid, and trimethoprim-sulfamethoxazole. The lavage solution may be based on or contain antiseptic acting chemicals such as for example H₂O₂, Chlorhexidine (CHG) and tinctures of CHG.

In a manner similar to intra-vascular infusion solutions, the lavage solution desirably is supplied from a soft bag container. FIG. 7 schematically illustrates a lavage container 80 that holds the supply of lavage solution that is introduced during the pharyngeal lavage step. As schematically shown therein, the tracheal tube 40 has a subglottic (or hypopharyngeal when using a double cuffed tracheal tube) lavage/suction port 43, and a connector 81 is disposed between container 80 and the lavage/suction port 43. The lavage solution container 80 desirably is provided in the form of a soft-bag solution container 80 with an integrated tube 82 that desirably is fused or welded with bag 80 at site 83. A regulating wheel 84 desirably is provided for dosing of the flow of lavage solution and desirably is disposed between the lavage solution container 80 and the site of the connector 81.

Removal of Lavage Solution

In accordance with the present disclosure, a high-volume suctioning device is used to remove from the intubated patient such dislodged and washed out residual colonized material that has been mobilized during the high volume infusion of the lavage solution.

The lavage solution and the material that has been washed away by the lavage solution can be removed via large bore, suctioning catheters. Such oro-pharyngeal suctioning catheters and naso-pharyngeal suctioning catheters are configured with sufficiently soft and pliable distal ends so as to be respectively suited for atraumatic intra-nasal insertion and suctioning and atraumatic naso-pharyngeal insertion and suctioning. Moreover, such catheters are designed to be able to reach sufficiently deep into the hypo-pharynx and especially into the naso-pharynx to remove the residual colonized material that has been mobilized during the high volume infusion of the lavage solution. It is desirable to apply the suction at the most distal end possible in order to maximize removal of most of the lavage solution and carried debris.

Typically, the duration of time needed to complete one cycle of introducing the lavage solution and removing the lavage solution is anticipated to be on the order of about three minutes.

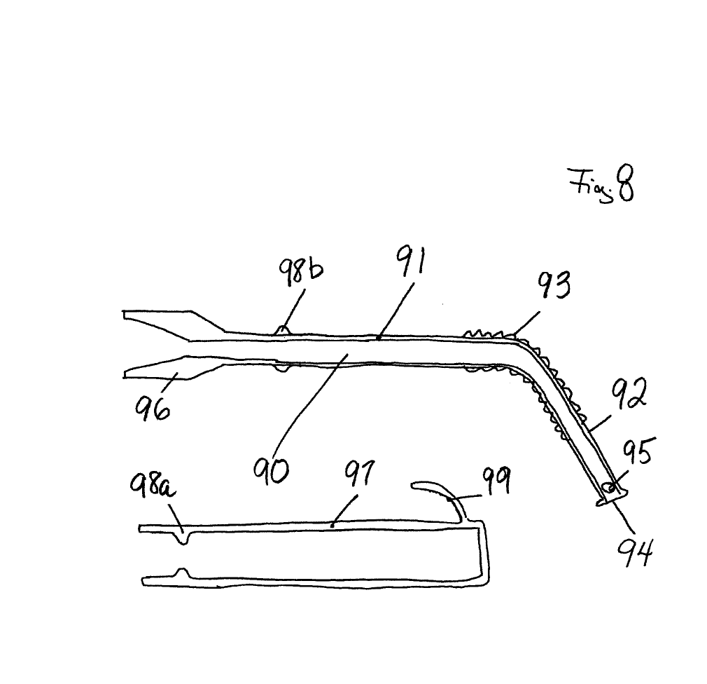

FIG. 8 schematically illustrates an embodiment of an oro-naso pharyngeal suction catheter suitable for performing the suctioning of the lavage solution and thereby removing the lavage solution from the patient after the lavage solution has been used to flush pathogenic contaminants out from the oro-naso cavities. As schematically shown in FIG. 8, an embodiment of an oro-naso pharyngeal suction catheter 90 desirably is configured in a typical Yankauer-like configuration and can be so pre-shaped by being extruded or injection molded. The distal opening 94 is configured atraumatically and may carry additional lateral decompression openings 95.

Additionally, the proximal portion 91 of the catheter 90 desirably is provided with sufficient stiffness so as to be able to resist kinking. While the distal part 92 of the catheter 90 is sufficiently soft to provide atraumatic performance when in use in the patient. One way of imparting such differing measures of stiffness and softness to different portions of the catheter 90 is by a gradual reduction of wall thickness of the suctioning element 90 from its proximal portion 91 to its distal portion 92. Another way of imparting such differing measures of stiffness and softness to different portions of the catheter 90 is by making the catheter 90 of tubing of two different Shore hardnesses, ranging from high Shore hardness at the proximal portion 91 to low Shore hardness at the distal portion 91 where Shore hardness is defined to be a measure of the resistance of material to indentation by a spring-loaded indenter. The higher the Shore hardness number, which normally is for plastic or rubber materials, the greater the resistance of the material to denting. Moreover, as schematically shown in FIG. 8, still another alternative way of accomplishing the same result is by providing a corrugated section 93 within the Yankauer angle. Desirably, the corrugation folds are disposed in a ratio of about one third in the proximal straight portion 91 and two thirds within the beveled distal portion 92.

The catheter 90 desirably is configured for repeated use, and a connector 96 disposed at the proximal end of the catheter 90 desirably can be configured to mate with the connector of a standard oral care suctioning hand piece, thereby enabling the suctioning force to be controlled by an integrated thumb decompression opening or another suitable mechanism for regulating the suctioning pressure.

A tubular sheath 97 can be configured to receive therein the catheter 90 for initial packaging and for re-insertion for preservation and storage for subsequent repeated uses. Desirably, the catheter 90 (connected to a hand piece) can be inserted into the sheath 97 and retained therein by click-in mechanism, which can be provided by two ring-like prominent structures 98a, 98b, one ring 98a being configured internal to the sheath 97 and the other ring 98b being configured external to the catheter 90. The resiliently deformable ring structures 98a, 98b desirably provide enough retaining resistance so as to keep the catheter 90 plus any attached suctioning unit fixed and in place within the sheath 97 until desired to be withdrawn for use. The sheath 97 desirably can be equipped with a hook like part 99, which can be used to hang the sheath 97 and catheter 90 in the environs of the patient.

Alternatively a foil based sheath 97a can be used. The sheath 97a can be fixed to two ring-like structures 97b. During the suctioning maneuver, the retracted sheath 97a can be locked in position by structure 98b, which is prominently disposed on the catheter element 91. In the intermittent phases, the catheter protecting sheath 97a can be pulled towards the tube tip, to be unfolded to fully cover the tip, and be hung up by the hook 99 in the patient's environs.

In accordance with an alternative embodiment of the present disclosure, a hand-held oral suctioning unit desirably can be connected directly onto the connector element of the lumen 46 that communicates with the lavage port 43 of the tracheal tube 40 in order to remove residual lavage solution from the subglottic space or the hypopharyngeal space. Similarly, in order to remove residual lavage solution from the subglottic space or the hypopharyngeal space, as shown schematically in FIG. 7 for example, a high volume suctioning device 72 desirably can be connected via the connector 81 directly to the lumen 62 that communicates with the port 63 of the NG tube 61 and/or to the lumen 46 that communicates with the flushing port 43 of the tracheal tube 40. The high volume suctioning device 72 desirably can be configured with its own disposable reservoir for receiving the residual fluids that are suctioned from these spaces and easily disposed of same.

Phases Between Lavage

In accordance with an embodiment of the present disclosure, the efficacy of the lavage that has been performed can be determining by appropriate testing. Such testing desirably includes the determination of the pharyngeal colonization density of bacteria in terms of the qualitative and quantitative status of pharyngeal surface colonization of bacteria.

Before a lavage maneuver is performed, the colonization status of the pharyngeal surfaces desirably can be determined by taking a direct specimen sample from the interior surfaces of the patient's cheeks for example. After analyzing the sample, the microbiological result can be reported to the healthcare provider, who based on the reported microbiological finding, then either can continue the regular lavage scheme, intensify the lavage scheme, change the scheme to a lavage solution with active, possibly antiseptic and/or antibiotic ingredients or intermittently apply an antiseptic oro-pharyngeal spray. An alternative to cultivation is the use of direct read indicators.

By closing the information loop between the healthcare provider and microbiological result, the present disclosure enables the pharyngeal care to become a primarily preventive measure, aiming consequently at the idea of monitoring and actively controlling the growth of bacteria within the pharynx and its adjunct cavities.

Gel

In accordance with the present disclosure, after removing the lavage solution, a space filling gel seal desirably is installed within the subglottic space or hypopharyngeal space and is composed of a preferably self-degrading physiologic or natural substance. Once the subglottic space or the hypopharyngeal space has been cleared from the remaining lavage solution and associated dislodged materials, the subglottic space and/or the hypopharyngeal space can be filled up with a space occupying gel. The gel provides an additional barrier against pathogens becoming resident near the tracheal balloon seal and improves the seal efficacy of the sealing cuff 50 or the esophageal balloon 60, as the case may be.

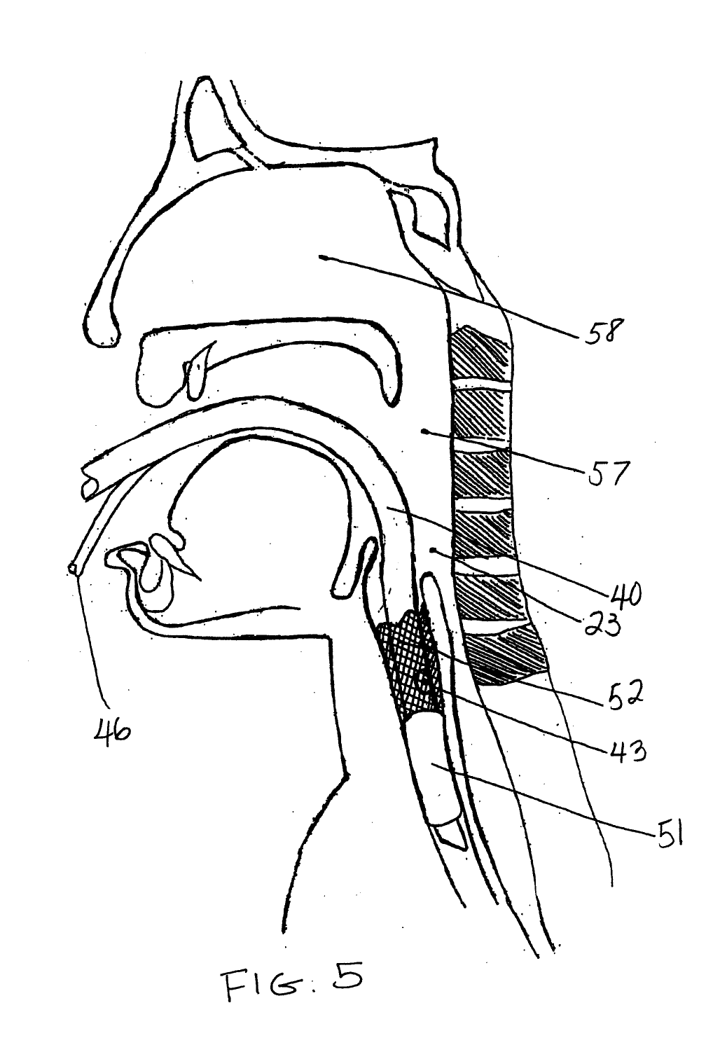

The gel can be injected via the same integrated lumen device 46, 62 that provided the conduit for introducing the lavage solution. As schematically shown in FIG. 5 for example, the gel desirably can be injected via the lavage conduit 46 and port 43 into the subglottic space to form a mass 52. The gel mass 52 prevents pharyngeal secretions from pooling directly above the cuff 51, thereby preventing fulminant aspiration of subglottic pooled material due to a sudden pressure drop in the balloon 51 that might be caused by a thoracic pressure drop. The gel mass 52 is removed in the subsequent lavage procedure and so is washed into the oral and nasal spaces 57 and 58. Desirably, the gel's initial viscosity should be set in way that a water column of 50 to 100 cm H2O, which can be effected by elevation of the connected rinsing fluid above the outlet level, suffices to remove the gel from inside the patient the fluid delivering lumen 46 of the catheters.

The gel desirably is made from a self degrading material that is based on a physiologic, body own substance (e.g., hyaluronic acid), which spontaneously degrades over time, and goes from a high viscosity state to a low viscosity state. The composition of the gel can be: Hyaluronic acid, sodium salt (HA), or crosslinked HA, or chitosan glycolate (CG), or a combination of HA and CG, or a combination of HA with other bioresorbable substances (HA/Polyglycolic acid, HA/polyvinyl pyrrolidone (PVP), HA/Dextran, HA/gums). The composition of the gel also can include Xylitol, which has a bacteriostatic effect on pharyngeal flora, thereby preventing formation of periodontal plaque, which is a VAP relevant reservoir.

Repetition Intervals

In accordance with the present disclosure, the successive steps of pharyngeal lavage, removal of the dislodged and washed out residual colonized material from the intubated patient by high-volume suctioning and optionally installing a space filling gel seal within the subglottic space or the hypopharyngeal space, can be repeated intermittently. As embodied herein, the repetition interval is suggested as either once per day or once per shift. However, adjustment of the interval can be made dependent on a number of factors, which include but are not limited to the condition of the patient.

Gastric contents pose the major contamination factor to be suppressed during the phases between the pharyngeal lavage procedures. The seal efficacy of the esophageal seal 60 significantly influences the achievable degree of pharyngeal colonization reduction. Thus, maintaining the esophageal seal 60 during periods between the high volume lavage improves VAP reduction.

Sealing Element Requirements

The general dependency between intra-thoracic pressure and intra-cuff pressure, under dynamical breathing conditions, can be described as follows. Inspiration is initiated by a volume increase of the intra-chest cavity. This increase is driven by an elevation of the chest apparatus by a muscular contraction of the intercostal muscles and a lowering of the diaphragm by contraction of its muscular portions. Following the rule pressure times volume equals a constant (PV=constant), the increase of chest volume results in a decrease of intra-chest pressure. Because the tracheal balloon seal 51 and the esophageal balloon seal 60 reside inside the chest cavity, the filling pressure of each balloon directly communicates with the pressure inside the chest cavity, and that pressure inside the chest cavity defines the force acting transmurally on the balloon seals 51, 60.

In spontaneous supported ventilation, in order to mobilize volume from the supplying ventilator circuit and make it flow into the lungs, the patient must overcome an isometric workphase. This is the work that the patient must do in order to overcome the initial elastance of the lung tissue before the lung volume begins to increase and air flow begins to flow toward the lungs. The work of breathing that is performed during this initial isometric contraction of the intercostal and diaphragmatic muscles causes a pressure drop in the sealing balloon. Pressure fluctuations due to changes of thoracic volume in the spontaneously ventilated patient or the supportedly ventilated patient can be expected in the magnitude of about 5 to about 15 cm H₂O.

In state-of-the-art intubation, the tracheal cuff element 51 is placed within the thoracic cavity, thus being exposed to alternating intra-chest pressures generated by the patient, being the driving force in respiration. With the increase of the intra-chest volume (elevation of the chest, lowering of the diaphragm), representing the onset point of breathing, intra-chest (intra-thoracic) pressure drops. Accordingly, the trans-mural forces resting on the intubated trachea are reduced, allowing for a certain dimensional (cross-sectional) increase of the trachea, leading to a decrease of cuff-filling pressure. The immediate and direct communication of patient chest mechanics with intra-cuff pressure and the resulting continuous fluctuation of cuff pressure critically affect the cuff seal.

As shown in FIG. 4, the pharyngeal lavage procedure is preferably performed with the patient in the supine position, whereby the liquid column installed above the tracheal tube cuff seal 51 (referred to vocal cord level 18), may reach a maximum height A of 10 to 15 cm of water in an adult patient. The cuff 51 on the tracheal tube 40 that delivers the lavage solution must be designed to enable a consistent seal performance under alternating intra-chest and intra-tracheal pressure conditions, which are described above.

In addition to withstanding the added pressure differential that might be created by the intermittent changes of intra-cuff/balloon pressure caused by chest movement during regular ventilator assisted breathing, the balloon seals 50, 60 that seal off the lumens of the organs to define the created pharyngeal compartment must be configured to withstand the added pressure of sufficiently large amounts of lavage solution that will reach and completely fill the maxillary, the ethmoidal and the frontal sinuses, without exposing the patient to the risk of tracheal aspiration of the installed lavage solution or to the risk of lavage solution descending into the stomach. The balloon seals 50, 60 also must be configured to withstand the added pressure differential that might be created by the possible patient response (e.g., repeated coughing and deep inspiratory chest movement following repeated coughing) during the lavage maneuver and during the suctioning maneuver.

The performance of the sealing balloon elements 50, 60 under the above mentioned conditions is believed to be largely defined by: (1) the dimensional ratio of the sealing balloon element to the dimension of the lumen to be occluded, (2) the properties of the material that forms the balloon's wall, and (3) the thickness of the balloon's wall.

The column of lavage solution established in an adult can be estimated in the range of about 8 to about 15 cm. Thus, the effective force resting on the balloon seal 51 can be assumed to be on the order of approximately 8 to about 15 millibar (mbar). Seal 51 therefore needs to be designed to seal against the installed lavage solution at a filling pressure of at least about 15 mbar. An assumed cuff pressure drop from about 25 mbar (clinical standard) to about 15 mbar therefore must not result in a seal failure that would allow an aspiration of the installed lavage solution.

In the embodiment shown in FIG. 3, the dashed line X1 must be considered relevant to the seal, and since the exerted force of a maximum filling with lavage solution is comparable, the cuffs 50, 51 must withstand about 15 cm of water as well. The pressure fluctuations to be expected in the outer, enclosing, proximal balloon element 50 are in a significantly lower order than the pressures exerted inside the chest cavity. As shown in FIG. 3, since the so called subglottic portion of the lower airway is not located within the chest cavity and therefore not exposed to changes of intra-thoracic pressure, the clinically required filling pressure for a cuff in that position may be therefore reduced to about 15 mbar.

FIG. 4 also schematically shows the topographical situation defining the seal performance requirements of an NG tube being equipped with a sealing balloon 60 to provide a seal against a head of lavage solution in the pharynx and against passage of material contained in the stomach. Line X2 describes the topographically seal relevant deepest point. The expected maximum column of fluid installed above the esophageal placed balloon 60 is in the order of about 10 to about 15 cm of water. Due to the thoracic placement of the balloon 60 and its exposure to intra-chest pressure, chest movement activity of the patient can cause fluctuations of balloon filling pressure. In a patient receiving supported ventilation and contributing active work of breathing when performing a tidal breath, a decrease of thoracic pressure in the order of about 5 to about 10 cm of water would be expected. A clinically applied balloon filling pressure therefore should be in the order of about 25 to about 30 cm H₂O, assuming a maximum drop of about 10 cm of water due to a decrease in intra-thoracic pressure. This leaves a remaining filling pressure of about 15 cm H₂O.

In order to equip the sealing balloon elements 50, 51, 60 with the capability to withstand a certain fluid pressure at a balloon seal filling pressure that is about equal, the balloon element is preferably designed in a particular fashion. Desirably, micro-thin balloons are chosen and have a wall-thickness on the order of about 5 to about 20 micrometers. The micro-thin balloon should be made from materials such as polyurethane, polyethylene, polypropylene, PET, or material with similar mechanical properties, enabling such low wall thickness, while still providing sufficient strength to secure mechanical and geometrical stability during extreme ventilation situations.

Due to the high seal performance of tracheal tube cuffs made from micro-thin balloon material, as described for example in U.S. Pat. No. 6,526,977, which is hereby incorporated herein in its entirety by this reference, conventionally single cuffed tracheal tube designs also can be used in the present disclosure and still provide sufficient seal performance against the elevated volumes of pharyngeal lavage solution that are required in the herein disclosed pharyngeal care concept. For such micro-thin balloons 51, 60 enable reliable seal efficacy at filling pressures as low as about 15 mbar, which is sufficient to withstand a column of about 10 to about 15 cm of lavage solution being built up in the patient's pharynx.

A micro-thin balloon that is dimensioned as explained above so as to reside in an inflated state inside the trachea, larynx or esophagus generates folds in the wall. The presence of these folds results in a residual wall that can be unfolded when the organ's lumen expands or taken up by the infolding of the wall when the organ's lumen contracts. Such a so-called residual micro-thin balloon, which has a residual wall that is taken up by the formation of infolding, ensures that the balloon does not need to be expanded under perfusion critical filling pressures in order to maintain the seal around the organ's lumen. The presence of infolded balloon wall material ensures that changes of organ diameter or changes of cross-sectional circumference can be compensated by portions of the balloon's wall that move into or out of the invaginations created in the balloon's wall.

The dimensions of the micro-thin balloon should be chosen to provide sufficient infolded balloon wall, even under extreme caliber or circumference changes within the organ. The micro-thin balloon should be dimensioned to be larger than the lumen of the organ into which the balloon is to be placed. The diameter of the micro-thin balloon effecting the tracheal seal or laryngo-tracheal seal 51 should be 20 to 50 percent larger than the expected tracheal or laryngeal diameter. Due to the capability of the esophagus to expand considerably and the complex internal folding of the mucosa, the sealing micro-thin balloon 60 intended to reside in the esophagus should have a balloon circumference of 100 to 200 percent of the circumference of the internal organ surface of the esophagus in the resting state.

Balloon wall distension and balloon wall mechanics in micro-thin balloons have negligible effects. The combination of a membrane-like wall structure and the residuality attribute that is due to infolding in micro-thin balloons enables the user of a micro-thin balloon to determine the actually effected seal force by setting it equal with the barometrically measured pressure in the balloon. Thus, when using a micro-thin balloon to effect seal forces, the seal force that one can expect to be exerted onto the organ tissues can be set largely equal with the applied filling pressure inside the balloon.

The use of residual micro-thin balloons results in the lowest possible sealing force gradients in pressure sensitive organs like the trachea and the esophagus. Moreover, the combination of a membrane-like wall structure and the residuality attribute found in micro-thin balloons also enables the balloons to achieve nearly inertia-less rapid adjustment to changes in the organ's dimension or in the morphology of the organ's lumen. Prominent intra-luminal organ structures can be covered by the residual balloon membrane, maintaining equal transmural force exertion over the inner organ surface and preventing perfusion critical force peaks on such organ structures. The described residual micro-thin balloon membrane also has a high adhesiveness to the organ surface. Furthermore, the extreme light mass of micro-thin balloons and the swiftly responding micro-thin balloons are ideally suited for intra-organ force or pressure sensing.

In a conventional balloon occlusion, higher filling pressures distend the balloon element to the size required to seal the esophagus and produce an according deformation of the organ. However, according to the present disclosure, the described balloon 60 is designed so as to avoid any alteration of the configuration of the esophagus organ by merely filling out residual spaces left within the organ's lumen in the sense of a low-pressure tamponade. The result is an atraumatic, non-irritating, stationary seal.

Cuff 50 is arranged primarily more proximally on the tube shaft 40, and being positioned within the neck portion of the patient and thus largely outside the patient's chest cavity, the subglottic seal balloon 50 is not exposed to thoracic pressure changes. Moreover, the space occupying balloon 50 is placed largely within the larynx. The anatomical structure of the subglottic portion of the larynx is configured like a cartilage ring, which is a rigid circular closed structure and therefore does not undergo any diameter or dimensional changes under spontaneous or supported ventilation or extreme respiratory maneuvers as may occur after repeated coughing.

Cuff 51 is arranged sequentially on the shaft of tracheal tube 40 and is exposed to pressures inside the chest cavity. However, under-inflation of both cuffs 50, 51 does not leave a gap between the two balloon elements 50, 51. One advantage of this sequential array of regular distal cuff 51 and proximal subglottic sealing balloon 50 is the inflation and/or deflation filling of one minimizes the effect on the other. In both possible arrangements of tracheal and subglottic balloons (sequential and enclosing), the distal tracheal 51 cuff shields the subglottic balloon element 50 against respiratory pressure that is built up in the distal airways below the tracheal seal. This arrangement can prevent transmission of potentially traumatic or perfusion relevant pressures onto the subglottic balloon 50 and thence onto the laryngeal, pressure-sensitive structures. The shielding of the inner/distal cuff 51 also prevents critical drops of lower airway pressure (tracheal pressure) from being communicated into the larynx and causing a critical pressure drop in the balloon seal 50 there. Therefore, the subglottic seal balloon 50 is not exposed to the risk of immediate drops of filling pressure as would occur with tracheal tubes provided with a conventional, thick-walled single cuff. Thus, stability tends to be maintained in the cuffs filling pressure despite exposure of the cuff pressure to fluctuations in thoracic pressure.

In the present disclosure, the sealing balloon element 60 cooperates with the seal provided by the cuff(s) surrounding the tracheal tube 40 to define the boundary of the stable space of the pharyngeal compartment required for performing the disclosed lavage scheme. The particular design of the shaft of the NG tube 61 and the membrane defining the sealing balloon 60 meet the seal requirements under dynamic conditions, which include changing intra-thoracic pressures, changing trans-diaphragmatic pressures and the peristaltic contractions running from the upper to the lower sphincter of the esophagus organ, and thereby provide the necessary stability of the seal under pharyngeal high volume lavage.

While at least one presently preferred embodiment of the present disclosure has been described using specific terms, such description is for illustrative purposes only, and it is to be understood that changes and variations may be made without departing from the spirit or scope of the following claims.

What is claimed:

1. A method for reducing the incidence of VAP in mechanically ventilated, intubated patients, comprising:
maintaining the patient in the supine position;
separating the patient's respiratory tract from communication with the patient's digestive tract in a manner that has the pharynx segmented off as a separated compartment that can retain at least a 10 cm column of liquid above the subglottic space in the patient.

2. A method as in claim 1, further comprising:
introducing a lavage solution for pharyngeal lavage in sufficiently high volume so as to mechanically dislodge and wash out residual colonized material from the remote cranio-facial cavities.

3. A method as in claim 1, further comprising:
introducing a lavage solution for pharyngeal lavage in sufficiently high volume so as to fill the entire volume of the remote cranio-facial cavities.

4. A method as in claim 2, wherein:
the pharyngeal lavage step includes ensuring that the lavage solution reaches the frontal sinus cavity.

5. A method as in claim 2, wherein:
the lavage solution continues to be introduced until lavage solution appears at the corners of the patient's mouth or at the patient's nasal openings.

6. The method as in claim 2, wherein said lavage solution is introduced initially into the hypo-pharynx.

7. The method as in claim 2, wherein said lavage solution is introduced initially into the sub-glottic space.

8. The method as in claim 2, wherein said lavage solution is introduced in a flow that is maintained over a range of about 1.5 to about 3.5 milliliters per second.

9. The method as in claim 2, wherein said lavage solution is introduced in a flow that is maintained over a range of about 1.8 to about 2.5 milliliters per second.

10. A method as in claim 2, wherein:
the lavage solution is a saline-based solution.

11. The method as in claim 2, wherein the lavage solution includes an antibiotic composition.

12. The method as in claim 2, wherein the lavage solution includes an antiseptic composition.

13. The method as in claim 2, wherein:
the residual colonized material is mechanically removed from the patient's remote cranio-facial cavities without abrasively contacting the tissue of the cranio-facial cavities.

14. A method as in claim 2, further comprising:
removing residual lavage solution from the intubated patient via a lumen integrated with the trachea tube and through a port defined in the tube and disposed above the cuff.

15. A method as in claim 2, further comprising:
removing from the intubated patient such dislodged and washed out residual colonized material using a high-volume suctioning device.

16. A method as in claim 15, wherein:
the high-volume suctioning device is a Yankauer-type.

17. A method as in claim 15, further comprising:
installing a space filling gel seal within the subglottic space; and
wherein the steps of pharyngeal lavage, removal of the dislodged and washed out residual colonized material from the intubated patient by high-volume suctioning and installing a space filling gel seal within the subglottic space, are repeated successively and intermittently.

18. A method as in claim 1, wherein:
the step of mechanically separating the respiratory tract from communication with the digestive tract includes using a cuff disposed around the distal end of the tracheal tube with which the patient is intubated so as to provide a seal between the tube and the walls of the trachea.

19. A method as in claim 18, wherein:
the pharyngeal lavage step includes introducing the lavage solution via a lumen integrated with the trachea tube and through a port defined in the tube and disposed above the cuff and poised to discharge directly into the subglottic space.

20. A method as in claim 18, wherein:
the step of mechanically separating the respiratory tract from communication with the digestive tract includes using a balloon equipped probe inserted into the stomach for feeding and decompression, wherein the balloon is pressurized to provide an esophageal seal between the walls of the esophagus and the exterior of the probe.

21. A method as in claim 20, wherein:
using a radiopaque marker ring at the proximal end of the balloon to determine the correct anatomical placement of the balloon carrying segment of the probe.

22. A method as in claim 20, wherein:
the pharyngeal lavage step includes introducing the lavage solution via a lumen integrated with the probe and through a port defined in the probe and disposed above the balloon and poised to discharge directly into the hypopharyngeal space.

23. A method as in claim 16, further comprising the step of:
washing out the space filling gel seal into the pharynx with the high-volume lavage solution during the next pharyngeal lavage procedure.

24. A method as in claim 23, wherein:
the space filling gel seal is transformed from gel to liquid by introducing a specific enzyme for this purpose before the high-volume lavage solution is introduced for the next pharyngeal lavage procedure.

25. A method as in claim 2, further comprising the step of:
determining the efficacy of the lavage that has been performed, said determination including the determination of pharyngeal colonization density of bacteria in terms of the qualitative and quantitative status of pharyngeal surface colonization of bacteria.

26. A method as in claim 25, further comprising:
using said determined status of pharyngeal surface colonization of bacteria as a guide in determining whether during the lavage step to intensify the introduction of the lavage solution, modify the composition of the lavage solution or both intensify the introduction of the lavage solution and modify the composition of the lavage solution.

27. A system for reducing the incidence of VAP in a mechanically ventilated, intubated patient, comprising:
a first tube having a proximal end and a distal end opposite said proximal end, said first tube being configured for insertion into a patient's trachea and defining a first port disposed so as to be positioned adjacent the patient's hypo-pharynx when inserted into the patient's trachea;
a first lumen disposed within said first tube and having a distal end connected to said first port;
a first cuff surrounding said first tube and disposed between the distal end of said first tube and said first port, said first tube further defining a cuff port disposed between said first port and the distal end of said first tube;
a cuff lumen disposed within said first tube and having a distal end connected to said first cuff via said cuff port;
a second tube having a proximal end and a distal end opposite said proximal end, at least said distal end of said second tube being configured for insertion into the patient's esophagus, said second tube further defining a second cuff port disposed toward the distal end of said second tube;
a second cuff surrounding said second tube and disposed toward the distal end of said second tube;
a second cuff lumen disposed within said second tube and having a distal end connected to said second cuff via said second cuff port;
wherein said first and second tubes and said first and second cuffs are configured to cooperate so as to mechanically separate the patient's respiratory tract from communication with the patient's digestive tract in a manner that has the pharynx segmented off as a separated compartment that retains liquid; and
a first space filling gel seal disposed on the portion of the exterior of said first tube disposed in the vicinity of said first port and contacting said first cuff, said first gel seal including a self-degrading physiologic substance.

28. A system as in claim 27, wherein:
said first cuff has a diameter that is in a range of about 20 percent to about 50 percent larger than the expected diameter of the lumen of the trachea in which the cuff is to be disposed.

29. A system as in claim 27, wherein:
said second cuff has a diameter that is in a range of about 100 percent to about 200 percent larger than the expected diameter of the lumen of the esophagus in which the cuff is to be disposed.

30. A system as in claim 27, further comprising:
a lavage container configured for selectively storing and supplying lavage solution, said container being connected in communication with at least one of said first tube and said second tube.

31. A system as in claim 27, further comprising:
said second tube defining a second port disposed so as to be positioned adjacent the patient's supra-glottic space when said second tube is inserted into the patient's esophagus; and
a second lumen disposed within said second tube and having a distal end connected to said second port.

32. A system as in claim 31, further comprising:
a second space filling gel seal disposed on the portion of the exterior of said second tube in the vicinity of said second port and contacting said second cuff, said second gel seal including a self-degrading physiologic substance.

33. A system as in claim 27, further comprising:
a high-volume suctioning device configured to remove from the intubated patient, dislodged and washed out residual colonized material, said suctioning device being selectively connected to at least one of said first tube and said second tube.

34. A system as in claim 33, wherein:
said high-volume suctioning device is a Yankauer-type.
